(19) United States
(12) Patent Application Publication
(10) Pub. No.: US 2005/0271692 A1
(43) Pub. Date: Dec. 8, 2005

(54) Topical cosmetic compositions

(75) Inventors: Carla Gervasio-Nugent, New York, NY (US); Timothy Matthis Morgan, Victoria (AU); Barrie Charles Finnin, Victoria (AU); Barry Leonard Reed, Victoria (AU)

Correspondence Address: FOLEY AND LARDNER;SUITE 500, 3000 K STREET NW, WASHINGTON, DC, 20007, US

(73) Assignee: Cosmeceutic Solutions Pty. Ltd.

(21) Appl. No.: 11/014,916

(22) Filed: Dec. 20, 2004

Related U.S. Application Data
(63) Continuation of application No. PCT/AU03/00786, filed on Jun. 24, 2003.
(60) Provisional application No. 60/391,079, filed on Jun. 25, 2002.

(51) Int. Cl. A61K 7/00

(52) U.S. Cl. 424/401

(57) ABSTRACT

A topical cosmetic composition including: at least one water-soluble cosmetic agent; at least one oil-soluble cosmetic agent; and a dermal penetration enhancer, wherein topical application of the composition results in the delivery of the water-soluble and oil-soluble cosmetic agents into the stratum corneum as well as delivery of the oil-soluble cosmetic agent into the epidermis and dermis.

### FIELD OF THE INVENTION

The present invention relates to cosmetic compositions that can be applied topically for transdermal delivery. The invention also relates to methods for the transdermal delivery of cosmetic agents.

### BACKGROUND OF THE INVENTION

Conventional means for administering therapeutic or cosmetic agents (‘active agents’) to a human or animal are usually limited to some degree by biological, chemical, and physical barriers. Examples of physical barriers are the skin and various organ membranes that must be traversed before the agent reaches a target. Chemical barriers include pH variations, lipid bi-layers, and degrading enzymes. Both biologically and chemically active agents are particularly vulnerable to such barriers.

Many active agents can be applied topically and this provides a convenient mode of administration, particularly for cosmetic agents that are typically applied to an area of the skin that is affected by a skin condition. However, the effectiveness of topical application of an active agent depends on two major factors: a) percutaneous absorption and penetration; and b) bioavailability of the penetrated active agent to the target site in the skin.

For an active agent to be effectively applied topically, the agent needs to penetrate the stratum corneum (the outer layer of the skin that includes layers of terminally differentiated keratinocytes) into the epidermal layers, and then be distributed and bioavailable to the target sites to provide an effect.

Many cosmetic agents require routine application over an extended time period, and for this reason topical application is advantageous because the administration regime is relatively simple and can be achieved with a minimum of inconvenience. However, to maximise the effectiveness of the treatment, as much of the cosmetic agent as possible needs to be absorbed into the skin and when the agent is applied topically by applying a cream or lotion to the skin it is common for at least some of the active agent to be lost by rubbing off or evaporation.

A further problem with some topical cosmetic applications is that the cosmetic agent is systemically toxic. For example, retinoids that are used in the treatment of acne and methotrexate that is used in the treatment of psoriasis, are systemically toxic. To reduce the effects of the systemic toxicity of these agents it is preferable to limit systemic dispersion of the agents away from the localised site of pathology.

Accordingly, there is a need in the field of cosmetics for improvements in compositions and methods for the topical delivery of cosmetic agents.

### SUMMARY OF THE INVENTION

In a first form the present invention provides a topical cosmetic composition including:
at least one water-soluble cosmetic agent;
at least one oil-soluble cosmetic agent; and
a dermal penetration enhancer;
   wherein topical application of the composition results in the delivery of the water-soluble and oil-soluble cosmetic agents into the stratum corneum as well as delivery of the oil-soluble cosmetic agent into the epidermis and dermis.

In one particular form, the invention provides a topical cosmetic composition including:
a. a water-soluble cosmetic agent, and a first oil-soluble cosmetic agent;
b. a second oil-soluble cosmetic agent; and
c. a dermal penetration enhancer,
   wherein topical application of the composition results in the delivery of the water-soluble and first oil-soluble cosmetic agents into the stratum corneum as well as delivery of the second oil-soluble cosmetic agent into the epidermis and dermis.

The dermal penetration enhancer is preferably one or more sunscreen esters such as those selected from the formula: 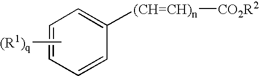
wherein
R¹ is hydrogen, lower alcohol, lower alkoxy, halide, hydroxy or NR³R⁴;
R² is a C₈ to C₁₈ alkyl,
R³ and R⁴ are each independently hydrogen, lower alkyl or R³ and R⁴ together with the nitrogen atom to which they are attached form a 5- or 6-membered heterocyclic ring;
n is 0 or 1,
q is 1 or2,
wherein when n is 0 and R1 is NR³R⁴, the NR³N⁴ is para-substitued

Particularly preferred dermal penetration enhancers are sunscreen esters selected from the group consisting of C₈ to C₁₈ alkylcinnamate, C₈ to C₁₈ alkylmethoxycinnamate, C₈ to C₁₈ alkyl salicylate and mixtures thereof. Most preferably the dermal penetration enhancer is padimate O or octyl salicylate.

As used herein, the term ‘cosmetic agent’ means any compound, mixture of compounds, or preparations derived therefrom that are intended to be placed in contact with external parts or with the mucosal membranes of an animal body. (especially a human body) with a view to cleaning, changing the appearance, protecting and/or keeping the body parts to which the agent is applied in good condition.

Cosmetic agents of the present invention would normally be applied topically to change the appearance of a particular skin area. Although the present invention is not limited in terms of skin areas to which the cosmetic agent can be applied, in many cases the agent will be applied to a visible skin area (such as the face) to change the appearance of the skin in that area.

For the purposes of the present invention a cosmetic agent can be distinguished from other physiologically active agents such as drugs that are intended for use in the diagnosis, cure, mitigation, treatment or prevention of disease. By way of example, for the purposes of the present invention a cosmetic agent may preferably be selected from one or more of: anti-ageing agents, anti-wrinkle agents, antioxidants, anti-scarring agents, phytoestrogens, isoflavones, coumarins, lip balms and antiseptic anti-acne agents.

A ‘water-soluble’ cosmetic agent is a cosmetic agent that generally has a water solubility greater than about 0.2 grams per litre at 25 degrees Celsius and atmospheric pressure. In contrast, an ‘oil soluble’ cosmetic agent is immiscible with water at 25 degrees Celsius and atmospheric pressure. In those unusual instances where a cosmetic agent has high solubility in both water and oil, for example, melatonin, for the practical purposes of this invention it should be considered as an ‘oil soluble’cosmetic agent. It is possible to conduct solubility tests using standard procedures to determine whether a particular cosmetic agent is water-soluble or oil-soluble.

Preferably the sunscreen ester dermal penetration enhancer is selected from C8 to C18 alkyl cinnamate, C8 to C18 alkyl methoxycinnamate or C8 to C18 alkyl salicylate. Most preferably the, sunscreen ester dermal penetration enhancer is selected from octyl para-methoxycinnamate or octyl salicylate. The sunscreen ester dermal penetration enhancer may also be a combination of two or more of the preferable enhancers.

The composition preferably includes a pharmaceutically acceptable carrier, and any of the carriers known in the art to be suitable for this purpose could be used. For example, as will be discussed in more detail later, the composition of the present invention may be applied as an aerosol and in that case the carrier is most preferably a volatile solvent.

The composition may include from about 0.1 to 50% by weight of the cosmetic agent, from about 1 to 10% by weight of the sunscreen ester dermal penetration enhancer, and from about 50 to 99% by weight of the pharmaceutically acceptable carrier.

The composition may also optionally include one or more additives selected from: a pharmaceutical compounding agent, co-solvent, surfactant, emulsifier, antioxidant, preservative, stabiliser and diluent.

Most preferably, the cosmetic agent is capable of diminishing, reducing or preventing the effects of one or more of the skin conditions including: the visible effects of aging, wrinkles, acne, age spots, scars (keloids), broken capillaries (telangiectases) and, includes compositions which also optionally cleanse the skin, preferably in the form of liquid compositions such as liquid soaps, lotions and solutions both additives and compositions for application to hair, scalp, nails, eyes or teeth.

In a second form the present invention provides a method of enhancing penetration of the skin by a cosmetic agent, the method including the step of applying to the skin a composition containing:
at least one water-soluble cosmetic agent;
at least one oil-soluble cosmetic agent; and
a dermal penetration enhancer;
   wherein topical application of the composition results in the delivery of the water-soluble and oil-soluble cosmetic agents into the stratum corneum as well as delivery of the oil-soluble cosmetic agent into the epidermis and dermis.

The sunscreen ester dermal penetration enhancer preferably acts to increase the penetration through the skin and mucous membrane of the cosmetic agent, in that way assisting in the delivery and retention of the oil-soluble cosmetic agent in particular into the epidermis and dermis. This may be by the percutaneous penetration of the agent across the epidermis and into the dermis and/or receptor fluid. Delivery of the water-soluble and oil-soluble cosmetic agents into the stratum corneum preferably results in the formation of a reservoir of the water-soluble and oil-soluble cosmetic agents within the stratum corneum such that no effective amount of the water-soluble and oil-soluble cosmetic agents are present in lower cell layers of the stratum corneum.

To determine whether or not an effective amount of the water-soluble and oil-soluble cosmetic agents are present in lower cell layers of the stratum corneum it is possible to carry out a tape strip test, whereby a layer of stratum corneum is removed with each adhesive tape strip, from which any cosmetic agent is extracted, prior to analysis. Preferably, using the method and composition of the present invention the lower cell layers of the stratum corneum contain no effective amount of the water-soluble and oil-soluble cosmetic agents when the region of the stratum corneum removed by tape strip numbers 15 to 20 is measured. More preferably, the lower cell layers of the stratum corneum are that region of the stratum corneum removed by tape strip numbers 19 to 20.

In a third form, the invention provides a method of reducing and/or preventing the effects of a skin condition, the method including the step of applying to an area of the skin that is affected by the skin condition a composition containing:
at least one water-soluble cosmetic agent;
at least one oil-soluble cosmetic agent; and
a dermal penetration enhancer;
   wherein topical application of the composition results in the delivery of the water-soluble and oil-soluble cosmetic agents into the stratum corneum as well as delivery of the oil-soluble cosmetic agent into the epidermis and dermis.

Preferably the skin condition is one or more of: the visible effects of aging, wrinkles, acne, age spots, scars (keloids), and broken capillaries (telangiectases) and includes compositions which also optionally cleanse the skin, preferably in the form of liquid compositions such as liquid soaps, lotions and solutions.

### DETAILED DESCRIPTION OF THE INVENTION

Preferred sunscreen ester dermal penetration enhancers for the purpose of the present invention are the 2-ethylhexyl (octyl) esters of p-methoxycinnamic acid, or salicylic acid. Whilst 2-ethylhexyl (octyl) esters are preferred, other long chain alkyl esters are also contemplated by the present invention.

Without being bound by any one particular theory as to the mode of action of the sunscreen ester dermal penetration enhancer, the present inventors believe that the sunscreen ester dermal penetration enhancer either alone or in combination with the pharmaceutically acceptable carrier can produce a reservoir effect in the skin or mucous membranes to which the composition is applied. The reservoir effect is thought to result from an enhancement of the ability of the skin or mucous membrane to both absorb and retain the cosmetic agents. The reservoir effect therefore allows the cosmetic agent to be absorbed and retained in larger amounts for longer periods of time than would be otherwise possible.

A preferred amount of the sunscreen ester dermal penetration enhancer is from about 1% to about 10% of the composition by weight. The compositions of the present invention preferably contain from about 0.1% to about 50%, preferably from about 1% to about 10% of the cosmetic agent by weight.

The dermal penetration enhancer of the present invention may also be used in conjunction with one or more other penetration enhancers that are known in the art. Examples of other useful penetration enhancers are disclosed in U.S. Pat. Nos. 4,537,776; 4,552,872; 4,557,934; 4,130,667; 3,989,816; 4,017,641; 4,628,078; and European Patent Application 0043738, each of which are incorporated herein solely for the purpose of providing details of prior art penetration enhancers.

Dosage forms for topical application of compositions of the present invention include solutions, lotions, creams, beach products, gels, sticks, sprays, pads, ointments, pastes, mousses, cosmetics, mouthwashes and aerosols. These product types may comprise several types of carrier systems including, but not limited to solutions, emulsions, gels and solids.

The cosmetic agent may be selected from any one or more of the following lists.

Anti-ageing agents include agents for treating wrinkles or preventing development thereof. Anti-ageing, anti-wrinkle, anti-skin atrophy and skin repair actives can be effective in replenishing or rejuvenating the epidermal layer. These actives generally provide these desirable skin care benefits by promoting or maintaining the natural process of desquamation. Examples of antiwrinkle and anti-skin atrophy actives include those selected from one or more of the list consisting of: alpha-lipoic acid, lycopene, ergothioneine, resveratol, grape seed extract, cis-urocaninic acid (see U.S. Pat. No. 5,620,680), compositions containing farnesol and/or bisabolol (see PCT Publication No. WO 00/62744), 2-amino-1,3-alkanediols, including but not limited to, 2-amino-1,3-octadecanediol, 2-N-acetylamino-1,3-octadecanediol, 2-N-octanoylamino-1,3-octadecanediol, 2-N-(2-hydroxyhexadecanoyl)amino-1,3-octadecanediol, 2-N-(2-hydroxydocosanoyl)amino-1,3-octadecanediol, 2-amino-1,3,4-octadecanetriol, 2-N-(2-hydroxyhexadecanoyl)amino-1,3,4-octadecanetriol, 2-N-hexanoylamino-1,3-octadecanediol, 2-N-octanoylamino-1,3,4-octadecanetriol (see U.S. patent Publication No. 20020006420 to Philippe et al.), ubiquinones and plastoquinones (coenzymes Qn), particularly coenzymes. Q9 and Q10, prasterone, DHEA, dehydroepiandrosterone, formulations containing one or more compounds chosen from the group consisting of sterols (such as zoosterols including cholesterol, dihydrocholesterol, 7-dehydrocholesterol, lanosterol, dihydrolanosterol, spongosterol and stellasterol, phytonterols including ergosterol, sitosterol, stigmasterol, fucosterol, brassicasterol and campesterol, mycosterols including ergosterol, fungisterol and zymosterol) and biochemical precursors thereof (such as mevalonic acid, famesol and squalane) in combination with one or more compounds including ubiquinones and derivatives thereof and plastoquinones and derivatives thereof (see U.S. Pat. No. 6,261,575), bertholletia extracts, betulinic acid, biotin, blackberry bark extract, blackberry lily extracts, black cohosh extract, blue cohesh extract, butanoyl betulinic acid, carboxymethyl 1,3 beta glucan, chalcones, chaste tree extract, clover extracts, coumestrol, daidzein, dang gui extract, darutoside, debromo laurinterol, 1-decanoyl-glycero-phosphonic acid, dehydrodicreosol, dehydrodieugenol, dehydroepiandrosterone, dehydroepiandersterone sulfate, dianethole, diosgenin, dodecanedioic acid, ergosterol, fennel extract, fenugreek seed extract, formononetin, forsythia fruit extract, genistein, genisteine, genistic acid, gentisyl alcohol, gingko bilboa extracts, ginseng extracts, ginsenoside (RO, R₆₋₁, R₆₋₂, R₆₋₃, R_{C}, R_{D}, R_{E}, R_{F}, R_{F-2}, R_{G-1}, R_{G-2}), 25-hydroxycholesterol, 7-hydroxylated sterols, hydroxyethyl isostearyloxy isopropanolamine, hydroxytetra methyl piperidinyloxy, hypotaurine, ibukijakou extract, isoflavones, isoflavone SG 10 (available from Barnet Products), kinetin, kohki extract, lectins, lichochalcone LF15 (available from Maruzen), licorice extracts, lignan, lumisterol, lupenes, luteolin, lysophosphitidic acid, naringenin, neotigogenin, o-desmethylangoiensin, oat beta glucan, oleanolic acid, placental extracts, pratensein, pregnenolone, pregnenolone acetate, pregnenolone succinate, soya extracts, spleen extracts, tachysterol, tigogenin, vitex extract, yam extract, yamogenin, zeatin, hyaluronic acid.

In a particularly preferred form of the invention, the anti-ageing agent is selected from one or more of the list consisting of ubiquinone, enzyme Q-10, alpha lipoic acid, lycopene and kinetin.

A phytoestrogen or isoflavone may be included as a cosmetic agent. The inclusion of a phytoestrogen or isoflavone may further increase the wrinkle regulating benefits of a composition by improving the thickness or structure of the dermis, or preventing further structural deterioration in the dermis. A safe and effective amount of phytoestrogen or isoflavone may be added to the compositions of the present invention, preferably from about 0.1% to about 10%, more preferably from about 1% to about 5%, of the composition.

In a particularly preferred form of the invention, the phytoestrogen or isoflavone is selected from one or more of the list consisting of genistein, daidzein, apigenin, phloretin, biochanin A, kaempferol, narinigenin, formononentin, ipriflavone, quercetin, chrysin and isoflavone SG10.

An anti-oxidant/radical scavenger may be included as a cosmetic agent. The inclusion of an anti-oxidant/radical scavenger may increase the wrinkle regulating benefits of a composition. A safe and effective amount of an anti-oxidant/radical scavenger may be added to the compositions of the present invention, preferably from about 0.1% to about 10%, more preferably from about 1% to about 5%, of the composition.

Anti-oxidants/radical scavengers such as melatonin, ascorbic acid (vitamin C) and its salts, tocopherol (vitamin E), tocopherol sorbate, other esters of tocopherol, butylated hydroxy benzoic acids and their salts, 6-hydroxy-2,5,7,8-tetramethylchroman-2-carboxylic acid (commercially available under the tradename Trolox™), gallic acid and its alkyl esters, especially propyl gallate, uric acid and its salts and alkyl esters, sorbic acid and its salts, the ascorbyl esters of fatty acids, amines (e.g., N,N-diethylhydroxylamine, amino-guanidine), sulfhydryl compounds (e.g., glutathione), and dihydroxy fumaric acid and its salts may be used.

In another preferred form of the invention the cosmetic agent is an agent for use in the treatment of venous blemishes that are a cosmetic problem often related to impaired peripheral microcirculation. Venus blemishes include broken capillaries, telangiectases, unesthitisms (for example cosmetic disfigurements or blemishes), scars and peripheral vasculopathies. Cosmetic agents for the purposes of this form of the invention include coumarins such as esculoside, esculetin, coumarin, extracts containing coumarins, and optionally, proanthocyanadins, such as proanthocyanadin A2, procyanidole oligomers extracted from Vitis vinifera and Camellia sinensis, and mixtures thereof, as described in U.S. Pat. No. 5,665,365 to Bombardelli et al.

Anti-age spot compositions of the present invention may be used to reduce or prevent the effects of hyper-pigmentary spots on the hands and other sun-exposed regions of the body. Compositions containing an alkanolamine such as ethylaminoethanol, methylaminoethanol, dimethylaminoethanol, isopropanolamine, triethanolamine, isopropanoldimethylamine, ethylethanolamine, 2-butanolamine, and mixtures thereof optionally containing adjunct ingredients such as sunscreens like microfine titanium dioxide, anti-oxidants, phytoestrogens and isoflavones may be used.

Suitable anti-acne agents may be selected from one or more of the list consisting of: phenoxyethanol, phenoxypropanol, phenoxyisopropanol, ethyl acetate, sebostats such as flavonoids and bioflavonoids; bile salts such as scymnol sulfate and its derivatives, deoxycholate, and cholate; allantoin; aloe extracts; barberry extracts; bearberry extracts; belamcanda chinensis; berberine; BIODERMINE (available from Sederma, located in Brooklyn, N.Y.); bioflavinoids; bisabolol; carrot extracts; cassin oil; clove extracts; citral; citronellal; cucumber extracts; ethyl hexyl monoglyceryl ether; ethyl 2-hydroxy undecanoate; farnesol; farnesol acetate; geranoil; glabridin; glyceryl monocaprate; grapefruit seed extract; gugu lipid; hesperitin; hinokitol; hops extract; hydrogenated rosin; lemon grass oil; linoleic acid; LIPACIDE C8CO (available from Seppic, located in Paris, France); mukurossi; neem seed oil; panthenol; 1-pentadecanol; peonia extract; peppermint extract; phelladendron extract; phloretin; PHLOROGINE (available from Secma); quercetin; red sandalwood extract; rosemary extract; rutin; sage extract; skull cap extract; siber hegner extract; siberian saxifrage extract; Sophora Extract (available from Maruzen); sunder vati extract; tea tree oil; thyme extract; trehalose 6-undecylenoate; 3-tridecene-2-ol; triclosan; white thyme oil; willow bark extract; wogonin; Ylang Ylang; zinc glycerolate; zinc linoleate; zinc oxide; zinc pyrithione; zinc sulfate; urea; triclosan, phenoxy ethanol and phenoxy proponol, ethylacetate, chlorhexidine and its derivatives such as chlorhexidine gluconate; sebostats such as flavinoids, prasterone, and antiseptic type antimicrobial agents and disinfectant type antibacterial agents such as chlorxylenol, chloroxylenol, octowynol and nonoxynol (see Martindale The Extra Pharmacopoeia 29^{th} Edition), urea, allantoin, povidone-iodine and phenol.

Lip balms compositions containing behenic acid or acexaminc acid, and antiseptic type antimicrobial agents and disinfectant type antibacterial agents such as chlorxylenol, chloroxylenol, octowynol and nonoxynol (see Martindale The Extra Pharmacopoeia 29^{th} Edition), urea, allantoin, povidone-iodine and phenol, menthol, camphor, triclosan, phenoxy ethanol, phenoxy proponol, ethylacetate, chlorhexidine and its derivatives such as chlorhexidine gluconate.

Anti-viral agents, for example behenic acid, C20 to C26 aliphatic alcohols including 1-docosanol, 1-tetracosanol, 1-hexacosanol.

Skin conditioning agents such as agents that smooth or soften the skin, selected from one or more of the list consisting of aloe vera extracts, Biocare SA (available from Amerchol); egg albumen; Flexan 130 (available from National Starch); Gatuline Lifting (available from Gattefosse); Pentacare HP (available from Pentapharm); Vegeseryl (available from Laboratories Serobioloques), candelilla wax, alpha bisabolol, aloe vera, Manjistha (extracted from plants in the genus Rubia, particularly Rubia Cordifolia), and Guggal (extracted from plants in the genus Commiphora, particularly Commiphora Mukul).

Skin whiteners, skin lighteners and agents for treating age spots, selected from one or more of the list consisting of dimethylaminoethanol, aloe extract, apple extract, arbutin, areca catechu L. extract, bamboo extract, bearberry extract, kojic acid, bletilla tuber, bupleurum falcatum extract, burnet extract, Burnet Power (available from Barnet Products), butyl hydroxy anisole, butyl hydroxy toluene, Chuanxiong, cola decaballo extract, Dang-Gui, deoxyarbutin, Fangfeng, fennel extract, ganodenna extract, gaoben, genistic acid and its derivatives, gentisyl alcohol, glycyrrhizinic acid, green tea extract, hyptis extract, lemon extract, licorice extract, Licorice P-TH (available from Bamet Products), linoleic acid, morus alba extract, mulberry root extract, parsley extract, phellinus linteus extract, pinon blanco extract, pinon negro extract, piri-piri extract, rose fruit extract, rucinol, Song-Yi extract, Sophora Powder (available from Barnet Products) and 4-thioresorein.

Astringent materials, for example hydrolysable tannins, phenolic acids associated with tannins, phenols associated with tannins, flavonoid compounds, natural extracts providing astringency, organic astringents and inorganic astringents (particularly salts of aluminium, zinc, iron (Ill), copper or silver).

Skin barrier repair actives are those skin care actives which can help repair and replenish the natural moisture barrier function of the epidermis. Nonlimiting examples of skin barrier repair actives include Alpha Lipid (available from Lucas Meyer); biotin; biotin esters; brassicasterol; caffeine; campesterol; canola derived sterols; Cennamides (available from Ennagram); Ceramax (available from Alban Muller); CERAMAX, (available from Quest, located in Ashford, England); CERAMIDE 2 and CERAMIDE HO3.™. (both available from Sederma); CERAMIDE II (available from Quest); CERAMIDE Ill and IIIB (both available from Cosmoferm, located in Deft, Netherlands); CERAMIDE LS 3773 (available from Laboratories Serobiologiques); CERAMINOL (available from Inocosm); Cerasol and Cephalip (both available from Pentapharm); cholesterol; cholesterol hydroxystearate; cholesterol isostearate; 7 dehydrocholesterol; DERMATEIN BRC and, DERMATEIN GSL (both available from Hormel); ELDEW CL 301 AND ELDEW PS 203 (both available from Ajinomoto); Fitobroside (available from Pentapharm); galactocerebrosides; Generol, 122 (available from Henkel); glyceryl serine amide; hydroxyethyl isostearyl isopropanolamine; lanolin; lanolin alcohols; lanosterol; lauric acid N laurylglucamide; lipoic acid; N-acetyl cysteine; N-acetyl-L-serine; N-methyl-L-Serine; Net Sterol-ISO (available from Barnet Products); palmitic acid; panthenol; panthetine; PHYTOSPHINGOSINE (available from Gist Brocades, located in King of Prussia, Pa.); PSENDOFILAGGRIN (available from Brooks Industries, located in South Plainfield, N.J.); QUESTAMIDE H (available from Quest); serine; sigmasterol; sitosterol; soybean derived sterols; sphingosine; sphingomylinase; S-lactoyl glutathione; stearic acid; Structurine (available from Silah); SUPER STEROL ESTERS (available from Croda); thioctic acid; THSC CERAMIDE OIL (available from Campo Research); trimethyl glycine; tocopheryl nicotinate; vitamin D₃; Y2 (available from Ocean Pharmaceutical).

Cosmetic soothing actives can be effective in preventing or treating inflammation of the skin. The soothing active enhances the skin appearance benefits of the present invention, e.g., such agents contribute to a more uniform and acceptable skin tone or color. The exact amount of anti-inflammatory agent to be used in the compositions will depend on the particular anti-inflammatory agent utilized since such agents vary widely in potency. Nonlimiting examples of cosmetic soothing agents include the following categories: absinthium, acacia, aescin, alder buckthorn extract, allantoin, aloe, arnica, astragalus, astragalus root extract, azulene, baikal skullcap, baizhu, balsam canada, bee pollen, BIOPHYTEX (available from Laboratories Serobiologiques), bisabolol, black cohosh, black cohosh extract blue cohosh, blue cohosh extract, boneset, borage, borage oil, bromelain, calendula, calendula extract, Canadian Willowbark Extract (available from Fytokem), candelilla wax, Cangzhu, canola phytosterols, celery seed, celery stem extract, CENTAURIUM (available from Sederma), centaury extract, chamazulene, chamomile, chamomile extract, chaparral, chaste tree, chaste tree extract, chickweed, chicory root, chicory root extract, chirata, chishao, collodial oatmeal, comfrey, comfrey extract, CROMOIST CM GWCAN (available from Croda), darutoside, dehurian angelica, devil's claw, divalent metals (such as, magnesium, strontium, and manganese), doggrass, dogwood, Eashave (available from Pentapharm), eleuthero, ELHIBIN (available from Pentapharm), ENTELINE 2 (available from Secma), ephedra, epimedium, esculoside; evening primrose, eyebright, Extract LE-100 (available from Sino Lion), Fangfeng, feverfew, ficin, forsythia fruit, Fytosterol 85 (available from Fytokem), ganoderma, gaoben, Gatuline A (available from Gattefosse), gentian, germanium extract, gingko bilboa extract, ginkgo, ginseng extract, goldenseal, gorgonian extract, gotu kola, grape fruit extract, guaiac wood oil, guggal extract, helenalin esters, henna, honeysuckle flower, horehound extract, horsechestnut, horsetail, huzhang, hypericum, immortelle, ipecac, job's tears, jujube, kola extract, LANACHRYS 28 (available from Lana Tech), lemon oil, lianqiao, licorice root, ligusticum, ligustrum, lovage root, luffa, mace, magnolia flower, manjistha extract, margaspidin, matricin, melatonin, MICROAT IRC (available from Nurture), mints, mistletoe, Modulene (available from Seporga), mono or diglucosides of glabridin, mono or diglucosides of gentisin, MTA (5′-deoxy-5′-methythioadenosine), mung bean extract, musk, N-methyl arginine, oat beta glucan, oat extract, orange, panthenol, papain, phenoxyacetic acid, peony bark, peony root, Phytoplenolin (available from Bio Botanica), phytosphingosine, Preregen (available from Pentapharm), purslane, QUENCH T (available from Centerchem), quillaia, red sage, rehmannia, rhubarb, rosemary, rosmarinic acid, royal jelly, rue, rutin, sandlewood, sanqi, sarsaparilla, saw palmetto, SENSILINE (available from Silab), SIEGESBECKIA (available from Sederma), stearyl glycyrrhetinate, Stimutex (available from Pentapharm), storax, strontium nitrate, sweet birch oil, sweet woodruff, tagetes, tea extract, thyme extract, tienchi ginserig, turmeric, urimei, ursolic acid, white pine bark, witch hazel xinyi, yarrow, yeast extract, yucca, and mixtures thereof.

Sebum stimulators can increase the production of sebum by the sebaceous glands. These skin care actives are especially useful for post-menopausal women who are sebum deficient. Examples of sebum stimulating actives include bryonolic acid, completech MBAC-DS, dehydroepiandrosterone (also known as DHEA), orizanol and mixtures thereof.

Sebum inhibitors can decrease the production of sebum by the sebaceous glands. Examples of sebum inhibiting actives include aluminium hydroxy chloride, ASEBIOL (available from Laboratories Serobiologiques), BIODERMINE (available from Sederma), cucumber extracts, gugulipiu, Lichochalcone LR 15 (available from Maruzen), phloretin, PHLOROGINE (available from Secma), S-carboxylmethyl cysteine, sepicontrol AS, zincidone (UC1B), and mixtures thereof.

As mentioned previously, any of the aforementioned cosmetic agents can be determined as being either water-soluble or oil-soluble using standard solubility testing procedures.

In one preferred form of the invention the water-soluble cosmetic agent is one or more of hydroxy acids, pyrollidone carboxylic acids, skin whiteners, astringents and water-soluble wrinkle-reducing agents.

A particularly preferred hydroxy acid is citric acid.

Also in a preferred form of the invention the first oil-soluble skin care agent is one or more of lipophilic antioxidants, ubiquinones, plastiquinones and oil-soluble skin barrier repair agents.

A particularly preferred antioxidant is an ubiquinone.

In a preferred form of the invention the second oil-soluble cosmetic agent is one or more of phytoestrogens, antioxidants, anti-scarring agents, isoflavones, prasterone, coumarins, behenic acid and retinoids.

Preferably the phytoestrogen or isoflavone is selected from the list consisting of genistein, daidzein, apigenin, phloretin, biochanin A, kaempferol, narinigenin, formononentin, ipriflavone, quercetin, chrysin and isoflavone SG10.

A particularly preferred phytoestrogen is genistein.

A particularly preferred antioxidant is melatonin.

A particularly preferred retinoid is retinol.

The pharmaceutically acceptable carrier may be any cosmetic vehicle that is toxicologically and pharmaceutically acceptable. Typical pharmaceutically acceptable carriers that can be used in compositions of the present invention include water, ethanol, acetone, isopropyl alcohol, stearyl alcohol, freons, polyvinyl pyrrolidone, propylene glycol, polyethlyene glycol, fragrances, gel-producing materials, mineral oil,stearic acid, spermaceti, sorbitan, monoleate, polysorbates, “Tweens,” sorbitol, methyl cellulose, petrolatum, a mineral oil (vaseline oil), which may be any petroleum based product; modified or unmodified vegetable oils such as peanut oil, wheatgerm oil, linseed oil, jojoba oil, apricot kernel oil, walnut oil, palm oil, pistachio oil, sesame oil, colza oil, cade oil, corn germ oil, peach kernel oil, poppyseed oil, pine oil, castor oil, soya oil, safflower oil, coconut oil, hazelnut oil, grapeseed oil, avocado oil, soy oil, sweet almond oil, calophyllum oil, castor oil, olive oil, sunflower oil, or animal oils such as whale oil, seal oil, menhaden oil, halibut liver oil, cod liver oil, cod, tuna, turtle tallow, horse's hoof, sheep's foot, mink, otter, marmot oil and the like; synthetic oils such as silicon oil such as dimethylpolysiloxane; alkyl and alkenyl esters of fatty acids, such as isopropyl esters of myristic, palmitic and stearic acids and fatty esters which are solid at room temperature; waxes such as lanolin wax, candelilla wax, spermaceti, cocoa butter, karite butter, silicon waxes, hydrogenated oils which are solid at room temperature, sucro-glycerides, oleates, myristates, linoleates, stearates, paraffin, beeswax, carnauba wax, ozokerite, candelilla wax, microcrystalline wax; fatty alcohols such as lauryl, cetyl, myristyl, stearyl, palmityl and oleyl alcohols; polyoxyethylated fatty alcohols; and wax esters, lanolin and its derivatives, perhydrosqualene and saturated esters, ethyl palmitate, isopropyl palmitate, alkyl myristates such as isopropyl myristate, butyl myristate and decyl myristate, hexyl stearate, triglyceride esters, triglycerides of octanoic and decanoic acid, cetyl ricinoleate, stearyl octanoate (Purcellin oil), fatty acids, polyhydric alcohols, polyether derivatives, fatty acid monoglycerides, polyethylene gylcol, propylene glycol, alkyl ethoxy ether sulfonates, ammonium alkyl sulfates, fatty acid soaps, and hydrogenated polyisobutene, and mixtures of waxes and oils.

The amount of the composition, and thus of the cosmetic agents to be administered, will be an effective amount for the desired result expected therefrom. This may be readily ascertained by the person skilled in the art utilising his ordinary skill and without undue experimentation.

Compositions of the present invention that are formulated as solutions typically include a pharmaceutically acceptable aqueous or organic solvent. Water is a typical solvent. Examples of suitable organic solvents include: ethanol, propylene glycol, butylene glycol, polyethylene glycol (200-600), polypropylene glycol (425-2025), glycerol, 1,2,4-butanetriol, sorbitol esters, 1,2, -6-hexanetriol, isopropanol, butanediol, and mixtures thereof. Preferably, these solutions contain from about 0.1% to about 50% of the cosmetic agent, more preferably from about 1% to about 20%; from about 1% to about 10% of the sunscreen ester penetration enhancer; and from about 1% to about 80% of an acceptable aqueous or organic solvent.

The composition in the form of a solution may be applied as an aerosol or spray using a dispensing apparatus that includes a shroud that keeps an actuator nozzle of the dispensing apparatus at a predetermined height above the site of application.

If the compositions of the present invention are formulated as an aerosol and applied to the skin as a spray-on, a propellant may also be added to the solution composition. A more complete disclosure of propellants that could be used can be found in Sagarin, “Cosmetics Science and Technology”, 2^{nd} Edition, Vol. 2, pp. 443-465 (1972).

A lotion can be made from a solution carrier system. Lotions preferably comprise from about 0.1% to about 50%, more preferably from about 1% to about 10%, of the cosmetic agent; from about 1% to about 20%, preferably from about 5% to about 10%, of an emollient; from about 1% to about 10% of the sunscreen ester dermal penetration enhancer; and from about 50% to about 90%, preferably from about 60% to about 80%, water.

Another type of product that may be formulated from a solution carrier system is a cream. A cream of the present invention would preferably comprise from about 0.1% to about 50%, more preferably from about 1% to about 10%, of the cosmetic agent; from about 5% to about 50%, preferably from about 10% to about 20%, of an emollient; from about 1% to about 10% of the sunscreen ester dermal penetration enhancer; and from about 45% to about 85%, preferably from about 50% to about 75%, water.

Yet another type of product that may be formulated from a solution carrier system is an ointment. An ointment may comprise a simple base of animal or vegetable oils or semi-solid hydrocarbons. Ointments may also comprise absorption ointment bases which absorb water to form emulsions. Ointment carriers may also be water soluble. An ointment may also comprise from about 2% to about 10% of an emollient plus from about 0.1% to about 2% of a thickening agent. A more complete disclosure of useful thickening agents can be found in Segarin, Cosmetics, Science and Technology, 2^{nd} Edition, Vol. 1, pp. 72-73 (1972).

Alternatively, the composition may be formulated as an emulsion having from about 1% to about 10%, preferably from about 2% to about 5%, of an emulsifier.

Emulsifiers may be nonionic, anionic or cationic. Suitable emulsifiers are disclosed in, for example, U.S. Pat. Nos. 3,755,560 and 4,421,769, and McCutcheon's Detergents and Emulsifiers, North American Edition, pages 317-324 (1986). Preferred emulsifiers are anionic or nonionic, although the other types may also be used.

Single emulsion skin care preparations, such as lotions and creams, of the oil-in-water type and water-in-oil type are well-known in the cosmetic art and are useful in the present invention. Multiphase emulsion compositions, such as the water-in-oil-in-water type (see U.S. Pat. No. 4,254,105) are also useful in the present invention. In general, such single or multiphase emulsions contain water, emollients and emulsifiers as essential ingredients.

Another emulsion carrier system useful in the topical pharmaceutical compositions of the present invention is a micro-emulsion carrier system. Such a system comprises from about 9% to about 15% squalane; from about 25% to about 40% silicone oil; from about 8% to about 20% of a fatty alcohol; from about 15% to about 30% of polyoxyethylene sorbitan mono-fatty acid (commercially available under the trade name Tweens) or other nonionics; and from about 7% to about 20% water. This carrier system is preferably combined with from about 1% to about 5% of the cosmetic agent.

If the compositions of the present invention are formulated as a gel or a cosmetic stick, a suitable amount of a thickening agent is preferably added to a cream or lotion formulation.

The compositions of the present invention may also be formulated as makeup products such as foundations.

The compositions of the present invention can optionally contain materials which are conventionally used in cosmetic compositions. Other conventional cosmetic or skin care additives the may be used include collagen, hyaluronic acid, elastin, hydrolysates, primrose oil, jojoba oil, epidermal growth factor, soybean saponins, mucopolysaccharides, and mixtures thereof may be used.

Other additives that may also be present in the compositions of the present invention include humectants, proteins and polypeptides, preservatives, alkaline agents, dyes, opacifiers, pigments, perfumes, thickeners and emulsifiers.

Compositions of the present invention may also include sunscreens other than the sunscreen ester dermal penetration enhancer(s), for example dioxybenzone, oxybenzone, padimate O, padimate A, aminobenzoic acid, cinoxate, diethanolamine p-methoxycinnamate, ethyl 4-[bis(hydroxypropyl)]aminobenzoate, ethylhexyl salicylate, glyceryl aminobenzoate, homosalate, the combination of lawsone and dihydroxyacetone, red petrolatum, and sulisobenzone.

### BRIEF DESCRIPTION OF THE FIGURES

In the accompanying figures:

FIG. 1 shows a plot of in vitro cumulative penetration of Melatonin across human epidermis.

FIG. 2 shows a plot of in vitro cumulative penetration of Genistein across human epidermis.

FIG. 3 shows a plot of in vitro Citric Acid content versus human skin tape number-10h after application.

FIG. 4 shows a plot of in vitro CoQ₁₀ content versus human skin tape number-10h after application.

### DETAILED DESCRIPTION OF EMBODIMENTS OF THE INVENTION

The invention will now be described with reference to the following non-limiting examples.

### Example 1

### Lip Balm

| Ingredients | % by weight |
|---|---|
| Polyethylene wax (MW 500) | 3.30 |
| Propylene glycol monoisostearate | qsf 100 |
| Octyl salicylate (penetration enhancer) | 5.00 |
| Behenic acid | 10.00 |
| Acexamic acid | 0.25 |
| Oxypropylenated (5 propylene oxides) | 12.00 |
| Lanolin wax | |
| Pyrrolidone carboxylic acid | 1.0 |
| Octacosanyl stearate | 7.60 |
| Hydroxyoctacosanyl hydroxystearate | 4.20 |
| Polybutene | 10.00 |

### Example 2

### Anti-Ageing Composition

| Ingredients | % by weight |
|---|---|
| PEG 1-glyceryl oleostearate + paraffin wax | 8.0 |
| Octyl salicylate (penetration enhancer) | 2.0 |
| Vaseline DAB | 2.8 |
| paraffin wax/paraffin | 1.8 |
| Paraffin oil (Mineral oil 5E, Shell) | 11.5 |
| Ceresin | 2.2 |
| Octyldodecanol | 10.0 |
| Genistein | 0.4 |
| Coenzyme Q.sub.9 | 0.2 |
| Coenzyme Q.sub.10 | 0.2 |
| Pyrrolidone carboxylic acid | 3.0 |
| Propylene glycol | 1.0 |
| Glycerol | 1.0 |
| Magnesium sulphate | 0.7 |
| Water, deionised | 54.4 |
| Total additives (perfume, preservative, | 0.8 |
| stabilisation) | |

### Example 3

### Anti-Wrinkle Composition

| Ingredients | % by weight |
|---|---|
| Cyclomethicone | 3.00 |
| Octyl salicylate (penetration enhancer) | 2.00 |
| Propyl paraben/laureth-7 | 0.75 |
| Mica/methicone | 0.01 |
| Red iron oxide/methicone | 2.70 |
| Yellow iron oxide/methicone | 2.70 |
| Black iron oxide/methicone | 2.70 |
| Titanium dioxide/cyclomethicone/ | 14.10 |
| dimethicone copolyol | |
| Zinc oxide/cyclomethicone/ | 5.00 |
| dimethicone copolyol | |
| Cyclomethicone/titanium dioxide/ | 3.80 |
| dimethicone copolyol/triethoxy caprylyl silane | |
| Spherical silica | 0.15 |
| Nylon-12 | 1.00 |
| Boron nitride | 1.05 |
| Titanium dioxide/methicone | 1.00 |
| Dimethicone | 7.25 |
| Cyclomethicone | 5.80 |
| Pyrrolidone carboxylic acid | 1.0 |
| Tribehenin | 0.10 |
| Retinyl palmitate | 0.01 |
| Genistein | 0.1 |
| Tocopheryl acetate | 0.01 |
| Aloe extract | 0.01 |
| Dimethicone | 1.50 |
| Polyglyceryl-4-isostearate | 1.50 |
| Cyclomethicone/dimethicone | 3.40 |
| Water | 26.50 |
| Salicylic acid/hydrolyzed vegetable protein | 0.50 |
| Methoxypropylgluconamide | 0.50 |
| Magnesium ascorbyl phosphate | 0.01 |
| Ethyl paraben/propylene glycol | 5.75 |
| Propylene glycol | 2.37 |
| Tetrasodium EDTA | 0.01 |
| Magnesium sulfate | 0.01 |
| Chamomile extract | 0.01 |
| Phytoclar | 0.01 |
| Soy protein | 3.00 |
| Cyclomethicone/dimethiconol | 2.00 |
| Methyl dihydrojasmonate | 0.25 |

### Example 4

### Anti-Wrinkle Composition

A composition for topical application to the skin containing the following ingredients by weight %:

| Ingredients | % by weight |
|---|---|
| Kinetin | 0.1 |
| Genistein | 0.2 |
| Pyrrolidone carboxylic acid | 0.5 |
| Octyl salicylate (penetration enhancer) | 2.0 |
| Oil phase | |
| Cetyl alcohol | 5.0 |
| Glyceryl monostearate | 15.0 |
| Sorbitan monooleate | 0.3 |
| Polysorbate 80 USP | 0.3 |
| Aqueous phase | |
| Methylcellulose 100 cps | 1.0 |
| Methyl paraben | 0.25 |
| Propyl paraben | 0.15 |
| Purified water | qsf 100 |

### Example 5

### Anti-Ageing Composition

A preferred makeup composition is described below.

| PHASE | INGREDIENTS | % |
|---|---|---|
| A | DI water | 20.70 |
| | Lipoic acid | 2.00 |
| | Ammonium hydroxide | 0.60 |
| | Pyrrolidone carboxylic acid | 0.2 |
| | Sodium chloride | 1.00 |
| B | 1,3-butylene glycol | 3.00 |
| | Octyl salicylate (penetration enhancer) | 2.00 |
| | Peg-30 dipolyhydroxystearate | 1.00 |
| | Genistein | 0.2 |
| | N-ethyl n-soyamorpholiniumethosulfate/water | 1.00 |
| | Glycerine | 2.00 |
| C | Titanium dioxide/isostearyl neopentanoate/ | 10.00 |
| | stearic acid/aluminum hydroxide | |
| | Polymethyl methacrylate | 2.00 |
| | Cyclomethicone/dimethicone copolyol | 5.00 |
| | Nylon-12 | 2.50 |
| | Isododecane | 3.50 |
| | Isohexadecane | 3.00 |
| | Vitamin e linoleate | 0.50 |
| | Sorbitan sesquioleate | 1.00 |
| | Cetyl dimethicone copolyol | 1.50 |
| | Cyclomethicone/quaternium-18 hectorite/ | 5.00 |
| | propylene carbonate | |
| | Lecithin | 0.50 |
| | Magnesium stearate | 0.50 |
| | Polyethylene | 2.00 |
| | Silica | 1.50 |
| | Silk powder | 1.00 |
| D | Titanium dioxide | 3.00 |
| | Cyclomethicone | 15.00 |
| | Talc | 0.50 |
| | Iron oxide yellow | 5.33 |
| | Iron oxide red | 1.42 |
| | Iron oxide black | 0.75 |
| E | Phenoxyethanol/isopropyl paraben/isobutyl | 1.00 |
| | paraben/butyl paraben | |

### Example 6

### Anti-Blemish (Acne) Composition

| Ingredients | % by weight |
|---|---|
| Water | 20.0 |
| Octyl salicylate (penetration enhancer) | 1.0 |
| Triclosan | 0.1 |
| Citric Acid | 0.02 |
| Melatonin | 0.1 |
| Ethanol | 40.0 |
| Glycerin | 30.0 |
| Fragrance | 0.1 |
| FD&C Blue 1 | 0.001 |
| Methylparaben | 0.8 |

### Example 7

### Anti-Blemish (Acne) Composition

| Ingredients | % by weight |
|---|---|
| Water | 20.0 |
| Octyl salicylate (penetration enhancer) | 1.0 |
| Triclosan | 0.1 |
| Citric Acid | 0.02 |
| Genistein | 0.1 |
| Ethanol | 40.0 |
| Glycerin | 30.0 |
| Fragrance | 0.1 |
| FD&C Blue 1 | 0.001 |
| Methylparaben | 0.8 |

### Example 8

### Broken Capillary Treatment Composition

| Ingredients | % by weight |
|---|---|
| Water | 20.0 |
| Octyl salicylate (penetration enhancer) | 2.0 |
| Citric Acid | 0.01 |
| Coumarin | 0.1 |
| Genistein | 0.1 |
| Ethanol | 40.0 |
| Glycerin | 30.0 |
| Fragrance | 0.1 |
| FD&C Blue 1 | 0.001 |
| Methylparaben | 0.8 |

### Example 9

### Liquid Soap Composition

| Ingredients | % by weight |
|---|---|
| Cetyl trimethyl ammonium chloride | 10.0 |
| Cocoamidopropyl amine oxide | 10.0 |
| Citric Acid | 0.02 |
| Lauryl myristyl monoethanol amide | 3.0 |
| Ethoxylated isodecyl alcohol | 20.0 |
| Octyl salicylate (penetration enhancer) | 1.0 |
| Genistein | 0.2 |
| Triclosan | 0.2 |
| Ethanol | 8.0 |

### Example 10

### Eye Gel

| Ingredients | % by weight |
|---|---|
| Carbomer | 1.5 |
| Cyclomethicone | 15.0 |
| Cyclomethicone-Dimethicone | 5.0 |
| Genistein | 0.4 |
| Hyaluronic Acid | 6.5 |
| L-ascorbic Acid | 5.0 |
| PEG-32 | 1.0 |
| Propylene Glycol | 2.5 |
| Retinyl Palmitate | 0.5 |
| Squalene | 2.0 |
| Tocopheryl Acetate | 0.8 |
| Total additives (perfume, | 0.8 |
| preservative, stabilisation) | |
| Water | qsf 100 |

### Example 11

### Hand And Body Moisturizer

| Ingredients | % by weight |
|---|---|
| Carbomer | 1.5 |
| Coenzyme Q.sub.10 | 0.2 |
| Cyclomethicone/dimethiconol | 3.0 |
| Genistein | 0.2 |
| Glycerin | 1.8 |
| Glyceryl monostearate | 11.0 |
| Glycolic acid | 0.2 |
| Hyaluronic acid | 8.0 |
| Mineral oil | 12.5 |
| Octyl salicylate | 2.5 |
| Peg-75 | 1.0 |
| Polyglyceryl-4-isostearate | 1.5 |
| Propylene glycol | 2.0 |
| Squalene | 1.5 |
| Total additives (perfume, | 0.8 |
| preservative, stabilisation) | |
| Water | qsf 100 |

### Example 12

### Smooth, Hydrating Face Mask

| Ingredients | % by weight |
|---|---|
| Ethanol | 10.0 |
| Genistein | 0.2 |
| Glycerin | 2.0 |
| Hyaluronic acid | 2.8 |
| Kinetin | 0.1 |
| Octyl salicylate | 2.5 |
| Paraffin oil(Mineral oil 5E, Shell) | 3.0 |
| PEG-75 | 1.0 |
| Polymethyl methacrylate | 2.0 |
| Polyvinyl alcohol | 11.0 |
| PVP/VA Copolymer | 4.0 |
| Xanthan gum | 0.5 |
| Water | qsf 100 |

### Example 13

### Smooth, Cleansing Mask

| Ingredients | % by weight |
|---|---|
| Butylene glycol | 3.0 |
| Ethanol | 10.0 |
| French Yellow Clay | 18.0 |
| Genistein | 0.4 |
| Glycolic acid | 0.2 |
| Kaolin | 10.0 |
| Octyl salicylate | 2.5 |
| Polysorbate 20 | 0.3 |
| Xanthan gum | 0.5 |
| Total additives (perfume, | 0.8 |
| preservative, stabilisation) | |
| Water | qsf 100 |

### Example 14

### Skin Diffusion Studies—Melatonin And Genistein

Formulation - with octyl salicylate

| Ingredients | % by weight |
|---|---|
| Propylene glycol | 6.0 |
| Cetearyl alcohol | 5.0 |
| Pyrollidine carboxylic acid (PCA) | 5.0 |
| Capric/caprylic triglycerides | 3.0 |
| Glyceryl stearate (non-self emulsifying) | 3.0 |
| Citric acid | 2.5 |
| Dimethicone (100 cs) | 2.0 |
| Melatonin | 2.0 |
| Octyl salicylate | 2.0 |
| PEG 40 stearate | 2.0 |
| Fragrance | 1.5 |
| Phenonip | 1.0 |
| Shea butter | 1.0 |
| Crill 3 | 0.5 |
| Genistein | 0.5 |
| Tocopherol | 0.5 |
| Xanthan gum | 0.35 |
| Coenzyme Q10 | 0.2 |
| Water | qsf 100 |

Control - without octyl salicylate

| Ingredients | % by weight |
|---|---|
| Propylene glycol | 6.0 |
| Cetearyl alcohol | 5.0 |
| Pyrollidine carboxylic acid (PCA) | 5.0 |
| Capric/caprylic triglycerides | 3.0 |
| Glyceryl stearate (non-self emulsifying) | 3.0 |
| Citric acid | 2.5 |
| Dimethicone (100 cs) | 2.0 |
| Melatonin | 2.0 |
| PEG 40 stearate | 2.0 |
| Fragrance | 1.5 |
| Phenonip | 1.0 |
| Shea butter | 1.0 |
| Crill 3 | 0.5 |
| Genistein | 0.5 |
| Tocopherol | 0.5 |
| Xanthan gum | 0.35 |
| Coenzyme Q10 | 0.2 |
| Water | qsf 100 |

Method

In vitro diffusion experiments were performed using stainless steel flow-through diffusion cells, using human epidermis maintained at 32° C. The receptor solution consisted of 20% EtOH in Milli-Q water. Eight cells for each condition were treated with 100 mg of appropriate donor phase. Samples were collected at appropriate time points and analysed by high performance liquid chromatography (HPLC).

Results

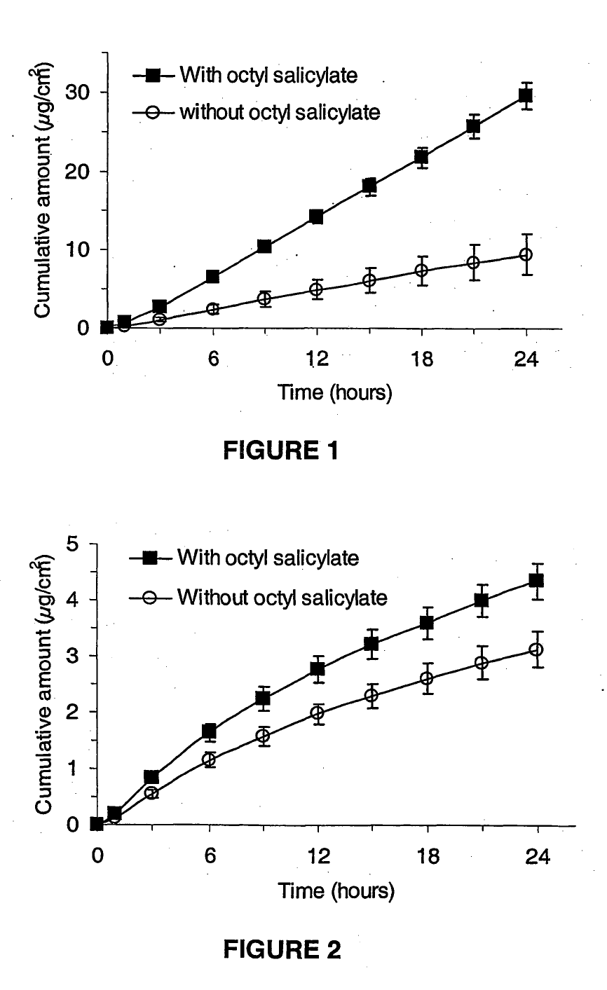

FIG. 1 and FIG. 2 shows the cumulative penetration of melatonin and genistein, respectively across human epidermis in vitro over 24 hours from o/w creams with or without octyl salicylate. Addition of octyl salicylate to an o/w cream was able to increase the cumulative amount of melatonin and genistein across human epidermis by 212% and 39%, respectively compared with the control formulation (p<0:05). Therefore, in vitro diffusion studies on the night-time moisturizer with octyl salicylate demonstrates the ability to enhance the delivery of both melatonin and genistein across human skin over a sustained period of time (24 hours) using a commercially and clinically relevant vehicle base.

### Example 15

### Skin Tape-Stripping Studies—Citric Acid And CoQ₁₀

Formulation - with octyl salicylate

| Ingredients | % by weight |
|---|---|
| Propylene glycol | 6.0 |
| Cetearyl alcohol | 5.0 |
| Pyrollidine carboxylic acid (PCA) | 5.0 |
| Capric/caprylic triglycerides | 3.0 |
| Glyceryl stearate (non-self emulsifying) | 3.0 |
| Citric acid | 2.5 |
| Dimethicone (100 cs) | 2.0 |
| Melatonin | 2.0 |
| Octyl salicylate | 2.0 |
| PEG 40 stearate | 2.0 |
| Fragrance | 1.5 |
| Phenonip | 1.0 |
| Shea butter | 1.0 |
| Crill 3 | 0.5 |
| Genistein | 0.5 |
| Tocopherol | 0.5 |
| Xanthan gum | 0.35 |
| Coenzyme Q10 | 0.2 |
| Water | qsf 100 |

Control - without octyl salicylate

| Ingredients | % by weight |
|---|---|
| Propylene glycol | 6.0 |
| Cetearyl alcohol | 5.0 |
| Pyrollidine carboxylic acid (PCA) | 5.0 |
| Capric/caprylic triglycerides | 3.0 |
| Glyceryl stearate (non-self emulsifying) | 3.0 |
| Citric acid | 2.5 |
| Dimethicone (100 cs) | 2.0 |
| Melatonin | 2.0 |
| PEG 40 stearate | 2.0 |
| Fragrance | 1.5 |
| Phenonip | 1.0 |
| Shea butter | 1.0 |
| Crill 3 | 0.5 |
| Genistein | 0.5 |
| Tocopherol | 0.5 |
| Xanthan gum | 0.35 |
| Coenzyme Q10 | 0.2 |
| Water | qsf 100 |

Method

Experiments were performed using full thickness human skin, after removal of the subcutaneous layer. Each skin sample was treated with 40 mg of appropriate donor phase. Serial sampling of the skin was completed at different time points after donor application, by tape stripping (3M Corp Adhesive tape) with a pressure load of 240 g/cm². Each tape strip removed approximately 1 layer of stratum corneum, and in each case, 20 tape strips were performed. Tape strips were extracted with methanol: chloroform and analysed by high performance liquid chromatography (HPLC).

Results

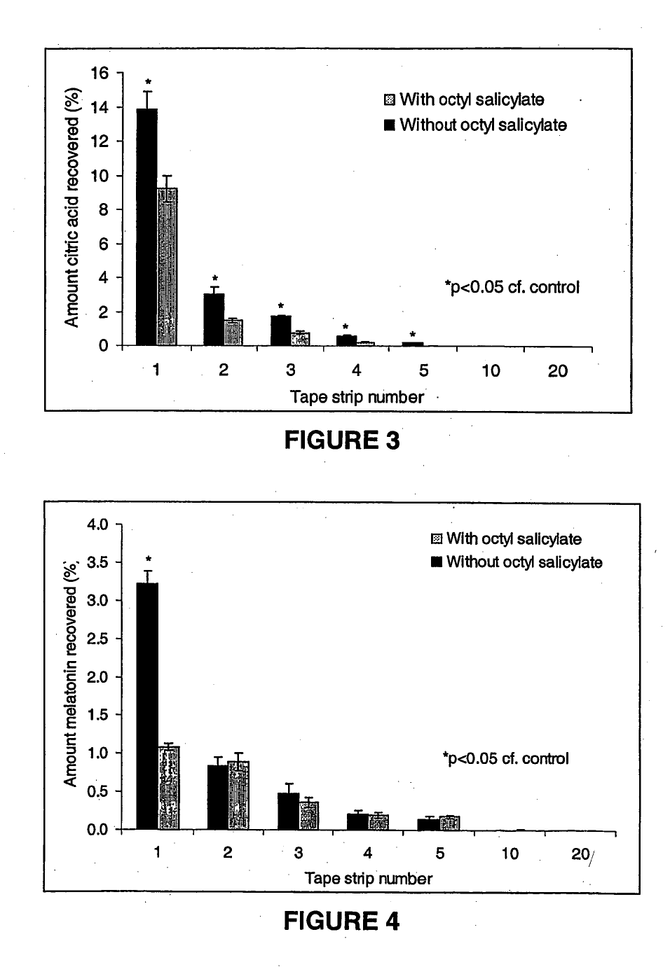

FIG. 3 and FIG. 4 shows the % amount of citric acid and CoQ₁₀ versus tape strip number-10 hours after application, respectively in human skin from o/w creams with or without octyl salicylate. Data for the 5 minute and 5 hour applications followed a similar pattern to those shown in FIGS. 3 and 4 but with lower absolute amounts in the 1-10 tape strip number range. Addition of octyl salicylate to an o/w cream was able to increase the amount of citric acid and CoQ₁₀ in the upper and middle regions of the stratum corneum. The targeted and enhanced delivery of citric acid to the stratum corneum is desirable given its intended purpose to act on this region of the skin as a mild keratolytic in order to smooth skin texture. The localization of CoQ₁₀ in the upper epidermis is due to its very high lipophilicity and poor water solubility. The in vitro skin tape stripping studies on the night-time moisturizer with octyl salicylate demonstrated the ability to enhance and target the delivery of both citric acid and CoQ₁₀ in the upper epidermis using a commercially and clinically relevant vehicle base.

Finally, it is understood that various other modifications and/or alterations may be made without departing from the spirit of the present invention as outlined herein.

1. A topical cosmetic composition including:
at least one water-soluble cosmetic agent;
at least one oil-soluble cosmetic agent; and
a dermal penetration enhancer;
wherein topical application of the composition results in the delivery of the water-soluble and oil-soluble cosmetic agents into the stratum corneum as well as delivery of the oil-soluble cosmetic agent into the epidermis and dermis.

2. A topical cosmetic composition as in claim 1, the composition including:
a water-soluble cosmetic agent, and a first oil-soluble cosmetic agent;
a second oil-soluble cosmetic agent; and
a dermal penetration enhancer,
wherein topical application of the composition results in the delivery of the water-soluble and first oil-soluble cosmetic agents into the stratum corneum as well as delivery of the second oil-soluble cosmetic agent into the epidermis and dermis.

3. A topical cosmetic composition as in either claim 1 or claim 2 wherein delivery of the water-soluble and oil-soluble cosmetic agents into the stratum corneum results in the formation of a reservoir of the water-soluble and oil-soluble cosmetic agents within the stratum corneum and no effective amount of the water-soluble and oil-soluble cosmetic agents are present in lower cell layers of the stratum corneum.

4. A topical cosmetic composition as in claim 3 wherein the lower cell layers of the stratum corneum contain no effective amount of the water-soluble and oil-soluble cosmetic agents when the region of the stratum corneum is removed and tape strip numbers 15 to 20 are measured.

5. A topical cosmetic composition as in claim 4 wherein the lower cell layers of the stratum corneum are that region of the stratum corneum removed by tape strip numbers 19 to 20.

6. A topical cosmetic composition as in either claim 1 or claim 2 wherein the dermal penetration enhancer is one or more sunscreen esters selected from the formula: 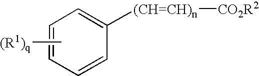
wherein
R¹ is hydrogen, lower alcohol, lower alkoxy, halide, hydroxy or NR³R⁴;
R² is a C₈ to C₁₈ alkyl,
R³ and R⁴ are each independently hydrogen, lower alkyl or R³ and R⁴ together with the nitrogen atom to which they are attached form a 5- or 6-membered heterocyclic ring;
n is 0 or 1,
q is 1 or 2,
wherein when n is 0 and R1 is NR³R⁴, the NR³N⁴ is para-substitued.

7. A topical cosmetic composition as in claim 6 wherein the dermal penetration enhancer is one or more sunscreen esters selected from the group consisting of C₈ to C₁₈ alkylcinnamate, C₈ to C₁₈ alkylmethoxycinnamate, C₈ to C₁₈ alkyl salicylate and mixtures thereof.

8. A topical cosmetic composition as in claim 7 wherein the dermal penetration enhancer is padimate O.

9. A topical cosmetic composition as in claim 7 wherein the dermal penetration enhancer is octyl salicylate.

10. A topical cosmetic composition as in claim 2 wherein the water-soluble cosmetic agent is one or more of hydroxy acids, pyrollidone carboxylic acids, skin whiteners, astringents and water-soluble wrinkle-reducing agents.

11. A topical cosmetic composition as in claim 10 wherein the hydroxy acid is citric acid.

12. A topical cosmetic composition as in claim 2 wherein the first oil-soluble cosmetic agent is one or more of lipophilic antioxidants, ubiquinones, plastiquinones and oil-soluble skin barrier repair agents.

13. A topical cosmetic composition as in claim 12 wherein the lipophilic antioxidant is an ubiquinone.

14. A topical cosmetic composition according to claim 2 wherein the second oil-soluble cosmetic agent is one or more of phytoestrogens, antioxidants, anti-scarring agents, isoflavones, prasterone, coumarins, behenic acid and retinoids.

15. A cosmetic composition according to claim 14 wherein the phytoestrogen or isoflavone is selected from the list consisting of genistein, daldzein, apigenin, phloretin, biochanin A, kaempferol, narinigenin, formononentin, ipriflavone, quercetin, chrysin and isoflavone SG10.

16. A topical cosmetic composition as in claim 14 wherein the phytoestrogen is genistein.

17. A topical cosmetic composition according to claim 14 wherein the retinoid is retinol.

18. A topical cosmetic composition as in claim 14 wherein the antioxidant is melatonin.

19. A topical cosmetic composition as in claim 2 wherein the water-soluble cosmetic agent is a mixture of citric acid and pyrollidone carboxylic acid, the first oil-soluble cosmetic agent is coenzyme Q10, the second oil-soluble cosmetic agent is a mixture of genistein and melatonin and the dermal penetration enhancer is octyl salicylate.

20. A topical cosmetic composition as in claim 2 additionally including an antiseptic, anti-acne or anti-blemish agent, or mixtures thereof.

21. A topical cosmetic composition as in claim 6 wherein the composition comprises from 0.1% to 10% by weight of dermal penetration enhancer.

22. A topical cosmetic composition as in either claim 1 or claim 2 further including a pharmaceutically acceptable carrier.

23. A topical cosmetic composition as in claim 2 in the form of a solution containing the cosmetic agents in an amount of from 0.1% to 50% by weight of the total cosmetic composition, the dermal penetration enhancer in an amount of from 0.1% to 10% by weight of the total cosmetic composition, and a cosmetically acceptable aqueous or organic solvent in an amount of from 1% to 80% by weight of the total cosmetic composition.

24. A topical cosmetic composition as In claim 23 wherein the cosmetic agents are present in an amount from 1% to 20% by weight of the total cosmetic composition.

25. A topical cosmetic composition as in claim 2 in the form of a lotion containing the cosmetic agents in an amount from 0.1% to 50% by weight of the total cosmetic composition, an emollient in an amount from 1% to 20% by weight of the total cosmetic composition, the dermal penetration enhancer in an amount from 0.1% to 10% by weight of the total cosmetic composition, and a cosmetically acceptable aqueous or organic solvent in an amount from 1% to 80% by weight of the total cosmetic composition.

26. A topical cosmetic composition as in claim 25 wherein the cosmetic agents are present in an amount from 1% to 20% by weight of the total cosmetic composition.

27. A topical cosmetic composition as in claim 25 wherein the emollient is present in an amount from 5% to 10% by weight of the total cosmetic composition.

28. A topical cosmetic composition as in claim 25 wherein the solvent is present in an amount from 60% to 80% by weight of the total cosmetic composition.

29. A topical cosmetic composition as in claim 2 in the form of a cream containing the cosmetic agents in an amount from 0.1% to 50% by weight of the total cosmetic composition, an emollient in an amount from 5% to 50% by weight of the total cosmetic composition, the dermal penetration enhancer in an amount from 0.1% to 10% by weight of the total cosmetic composition, and a cosmetically acceptable aqueous or organic solvent in an amount from 45% to 85% by weight of the total cosmetic composition.

30. A topical cosmetic composition as in claim 29 wherein the cosmetic agents are present in an amount from 1% to 20% by weight of the total cosmetic composition.

31. A topical cosmetic composition as in claim 29 wherein the emollient is present in an amount from 10% to 20% by weight of the total cosmetic composition.

32. A topical cosmetic composition as in claim 29 wherein the solvent is present in an amount from 50% to 75% by weight of the total cosmetic composition.

33. A topical cosmetic composition as in claim 2 in the form of an ointment containing the cosmetic agent in an amount from 0.1% to 50% by weight of the total cosmetic composition, an emollient in an amount from 2% to 10% by weight of the total cosmetic composition, a thickening agent in an amount from 0.1% to 2.5% by weight of the total cosmetic composition, the dermal penetration enhancer in an amount from 0.1% to 10% by weight of the total cosmetic composition, and a cosmetically acceptable aqueous or organic solvent in an amount from 45% to 85% by weight of the total cosmetic composition.

34. A topical cosmetic composition as in claim 33 wherein the cosmetic agents are present in an amount from 1% to 20% by weight of the total cosmetic composition.

35. A topical cosmetic composition as in claim 33 wherein the solvent is present in an amount from 50% to 75% by weight of the total cosmetic composition.

36. A topical cosmetic composition as in claim 2 in the form of an emulsion containing the cosmetic agents in an amount from 0.1% to 50% by weight of the total cosmetic composition, an emulsifier in an amount from 1% to 10% by weight of the total cosmetic composition, the dermal penetration enhancer in an amount from 0.1% to 10% by weight of the total cosmetic composition, and a cosmetically acceptable aqueous or organic solvent in an amount from 45% to 85% by weight of the total cosmetic composition.

37. A topical cosmetic composition as in claim 36 wherein the cosmetic agents are present in an amount from 1% to 20% by weight of the total cosmetic composition.

38. A topical cosmetic composition as in claim 36 wherein the emulsifier is present in an amount from 2% to 5% by weight of the total cosmetic composition.

39. A topical cosmetic composition as in claim 36 wherein the solvent is present in an amount from 50% to 75% by weight of the total cosmetic composition.

40. A topical cosmetic composition according to claim 36 wherein the emulsifier is anionic or non-ionic.

41. A topical cosmetic composition as in claim 2 in the form of a cosmetic gel or stick containing the cosmetic agents in an amount from 0.1% to 50% by weight of the total cosmetic composition, an emollient in an amount from 2% to 10% by weight of the total cosmetic composition, a thickening agent in an amount from 0.1% to 5% by weight of the total cosmetic composition, the dermal penetration enhancer in an amount from 0.1% to 10% by weight of the total cosmetic composition, and a cosmetically acceptable aqueous or organic solvent in an amount from 1% to 80% by weight of the total cosmetic composition.

42. A topical cosmetic composition as in claim 41 wherein the cosmetic agents are present in an amount from 1% to 20% by weight of the total cosmetic composition.

43. A topical cosmetic composition as in claim 41 wherein the solvent is present in an amount from 20% to 70% by weight of the total cosmetic composition.

44. A method of enhancing penetration of the skin by a cosmetic agent, the method including the step of applying to the skin a composition containing:
at least one water-soluble cosmetic agent;
at least one oil-soluble cosmetic agent; and
a dermal penetration enhancer;
wherein topical application of the composition results in the delivery of the water-soluble and oil-soluble cosmetic agents into the stratum corneum as well as delivery of the oil-soluble cosmetic agent into the epidermis and dermis.

45. A method as in claim 44 wherein the dermal penetration enhancer is one or more sunscreen esters selected from the formula: 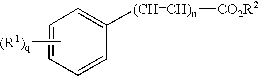
wherein
R¹ is hydrogen, lower alcohol, lower alkoxy, halide, hydroxy or NR³R⁴;
R² is a C₈ to C₁₈alkyl,
R³ and R⁴ are each independently hydrogen, lower alkyl or R³ and R⁴ together with the nitrogen atom to which they are attached form a 5- or 6-membered heterocyclic ring;
n is 0 or 1,
q is 1 or 2,
wherein when n is 0 and R1 is NR³R⁴, the NR³N⁴ is para-substitued.

46. A method of reducing and/or preventing the effects of a skin condition, the method including the step of applying to an area of the skin that is affected by the skin condition a composition containing:
at least one water-soluble cosmetic agent;
at least one oil-soluble cosmetic agent; and
a dermal penetration enhancer;
wherein topical application of the composition results in the delivery of the water-soluble and oil-soluble cosmetic agents into the stratum corneum as well as delivery of the oil-soluble cosmetic agent into the epidermis and dermis.

47. A method as in claim 46 wherein the dermal penetration enhancer is one or more sunscreen esters selected from the formula: 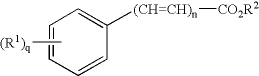
wherein
R¹ is hydrogen, lower alcohol, lower alkoxy, halide, hydroxy or NR³R⁴;
R² is a C₈ to C₁₈ alkyl,
R³ and R⁴ are each independently hydrogen, lower alkyl or R³ and R⁴ together with the nitrogen atom to which they are attached form a 5- or 6-membered heterocyclic ring;
n is 0 or 1,
q is 1 or 2,
wherein when n is 0 and R1 is NR³R⁴, the NR³N⁴ is para-substitued.

48. A topical cosmetic composition as in claim 1 substantially as hereinbefore described with respect to the accompanying examples.
